(19) United States
(12) Patent Application Publication
(10) Pub. No.: US 2024/0325707 A1
(43) Pub. Date: Oct. 3, 2024

(54) MULTILAYER MEDICAL TUBING WITH LOW SORBABILITY

(71) Applicant: CareFusion 303, Inc., San Diego, CA (US)

(72) Inventors: Zehra SEVINC, Long Grove, IL (US); Lawrence J. TRAINER, Murray, UT (US); Wantjinarjo SUWITO, West Linn, OR (US); Mark DOANE, North Fort Myers, FL (US); Anthony Charles GUEVARA, San Diego, CA (US)

(21) Appl. No.: 18/129,296

(22) Filed: Mar. 31, 2023

(51) Int. Cl. A61M 39/08; A61L 29/08

(52) U.S. Cl. CPC A61M 39/08; A61L 29/08; A61M 5/14

(57) ABSTRACT

Medical tube can have a continuous inner layer having a continuous outer layer thereon, in which the inner layer includes a polyolefin such as a polyethylene or polypropylene or a functionalized polyolefin. The outer layer can include a thermoplastic polymer such as one or more of, or a blend including, a thermoplastic polyurethane (TPU), a thermoplastic olefin (TPO), a thermoplastic elastomer (TPE), a styrene-containing thermoplastic elastomer (S-TPE), a polyolefin elastomer (POE), a styrenic blocking copolymer (SBC). Advantageously, the outer layer and/or the inner layer do not include polyvinyl chloride. Such tubing can be readily solvent bonded to medical connectors and used with infusion sets.

### TECHNICAL FIELD

The present disclosure generally relates to tubing and, in particular, to flexible medical tubing having low absorption of medicinal fluids and components in such fluids while being readily bondable to other medical components through solvent boding. Such tubing can be used for medical devices such as tubing for administration of medical fluid by infusion.

### BACKGROUND

Plastic tubing is extensively used in the medical field, particularly for patient analysis and treatment procedures. However, different and sometimes incompatible demands are required for medical tubing. For example, medical tubing should be strong yet soft or pliable, resist kinks, resist reacting with the fluid and avoid injecting adverse chemicals into fluid transported there through. But many plastic materials that have such characteristics tend to be inflexible. The inertness of many plastic materials that have such characteristics also make bonding such tubes difficult through solvent bonding. In many applications, however, medical tubing is pinched or clamped or used with infusion pumps that move fluid through the tubing by compressing the tubing. Such uses require the tubing to be flexible, easy to pinch and rebound quickly. Soft tubing such as polyvinyl chloride with plasticizer have been used in infusion sets for many years. Unfortunately, plasticized polymeric materials such as plasticized polyvinyl chloride can be sticky and can lead to occlusion and tearing of the tubing.

Hence, a continuing need exists for medical tubing that can address differing demands of medical applications.

### SUMMARY

Aspects of the subject technology relate to medical tubing comprising or consisting of a continuous inner layer having a continuous outer layer thereon. The inner layer can comprise or consist of a polyolefin and the outer layer can comprise or consist of a thermoplastic polymer selected among a thermoplastic polyurethane (TPU), a thermoplastic olefin (TPO), a thermoplastic elastomer (TPE), a styrene-containing thermoplastic elastomer (S-TPE), a polyolefin elastomer (POE), a styrenic blocking copolymer (SBC). Advantageously, the medical tubing has a bond strength that is at least 25% higher relative to medical tubing comprised of a monolayer of the thermoplastic polymer. It is believed that when the inner layer is more rigid, e.g., stiffer, than the outer layer, such a configuration facilitates solvent bonding of the medical tubing to other medical components.

The polyolefin inner layer can comprise or consist of a polyethylene or polypropylene or a functionalized polyolefin or a combination thereof wherein the functionalized polyolefin can be selected from a maleic anhydride modified polyethylene, a maleic anhydride modified polypropylene, a maleic anhydride modified plastomer, an amine functionalized polyolefin, or a combination thereof. The outer layer can comprise or consist of a thermoplastic polymer such as one or more of, or a blend including, a thermoplastic polyurethane (TPU), a thermoplastic olefin (TPO), a thermoplastic elastomer (TPE), a styrene-containing thermoplastic elastomer (S-TPE), a polyolefin elastomer (POE), a styrenic blocking copolymer (SBC). Advantageously, the inner layer is more rigid, e.g., stiffer, than the outer layer. Such a configuration facilitates solvent bonding of the medical tubing to other medical components. In addition, the outer layer and/or the inner layer advantageously do not include polyvinyl chloride.

The subject technology also relates to a method of manufacturing medical tubing by coextruding a continuous inner layer having a continuous outer layer directly thereon, wherein the inner layer comprises of consists of the polyolefin and the outer layer comprises or consists of the thermoplastic polymer. The method can further include extruding a tie layer between the continuous inner layer and continuous outer layer. The manufactured medical tubing can be formed transparent to visible light.

The subject technology also relates to a medical tube as referenced above bonded, via solvent boding, to a medical connector.

The subject technology also relates to an infusion set comprising the medical tubing bound to one or more medical connectors and a method of preparing an infusion set comprising the medical tubing bound to one or more medical connectors via solvent bonding.

Embodiments of the foregoing medical tubing and methods include one or more of the following features individually or combined. In some embodiments, the medical tubing can further comprises a tie layer between the continuous inner layer and continuous outer layer. The tie layer can comprise a maleic anhydride modified polypropylene, a maleic anhydride modified polyethylene, an ethyl vinyl acetate copolymer, or combinations thereof. Alternatively, the inner layer can directly contact the outer layer. Further, in other embodiments, the medical tubing can comprise an intermittent solvent bondable segment layer directly contacting the outer layer such as a thermoplastic polyurethane intermittent solvent bondable segment layer.

In still further embodiments, an infusion set can be prepared by applying a thin layer of solvent on one or both of a surface of the medical tubing and a surface of a medical connector, and holding the surface of the tubing and the surface of the medical connector together until a seal is formed therebetween. In some embodiments, the medical connector comprises an acrylic-based polymer.

Additional advantages of the subject technology will become readily apparent to those skilled in this art from the following detailed description, wherein only certain aspects of the subject technology are shown and described, simply by way of illustration. As will be realized, the subject technology is capable of other and different configurations, and its several details are capable of modifications in various other respects, all without departing from the subject technology. Accordingly, the drawings and description are to be regarded as illustrative in nature, and not as restrictive.

### BRIEF DESCRIPTION OF THE DRAWINGS

The accompanying drawing, which is included to provide further understanding and is incorporated in and constitute a part of this specification, illustrate disclosed embodiments and together with the description serve to explain the principles of the disclosed embodiments. In the drawings:

FIG. 1 illustrates an exemplary medical tube with a continuous inner and outer layer in accordance with aspects of the present disclosure.

FIG. 2 illustrates another exemplary medical tube including a continuous inner layer, a continuous outer layer thereon and a tie layer between the continuous inner layer and continuous outer layer in accordance with aspects of the present disclosure.

FIG. 3 illustrates a multi-material, polyolefin lined medical tube including a continuous inner polyolefin layer, a continuous outer layer and an intermittent solvent bondable segment layer directly contacting outer layer.

FIG. 4 is a bar graph showing the maximum load (Newtons) needed to break medical tubing bound to a medical connector. The graph compares medical tubing comprised of a monolayer (“mono”) of a polymeric material (control) to medical tubing having a polyolefin lined thermoplastic polymer and further compares such tubing to medical tubing prepared by alternative extrusion technology (AET).

FIG. 5 illustrates how certain variables affect extrusion of medical tubing.

FIG. 6 shows the effects of adhesion strength by adjusting certain variables during extrusion.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Aspects of the subject technology relate to medical tubing that can accommodate differing property requirements. Advanced material formulations and architectures are needed for the medical tubing to overcome technical challenges arising from conflicting design requirements for such tubing. For example, medical tubing should be capable of bonding to joints and other components to connect the tubing such as by solvent bonding due to the relative ease, convenience and costs of solvent bonding. Further medical tubing should simultaneously resist absorbing medicinal fluid and components therein with no significant changes to the tubing itself or any active pharmaceutical ingredient (API). That requires both an inert material for drug compatibility and a solvent responsive material for solvent bond-ability, which are typically conflicting requirements for a single material. Further there is a drive towards more environmentally friendly materials and to exclude polyvinyl chloride (PVC). Even though the tubing is expected to recover fast fully, it also needs to be rigid to enable cutting processes for manufacturability.

In some aspects, the subject technology relates to medical tubing including a continuous inner layer having a continuous outer layer thereon. Advantageously, the inner layer comprises or consists of a polyolefin which has low absorption characteristics such that the polyolefin inner layer resists absorbing medicinal fluid and/or components therein or affecting any active pharmaceutical ingredient (API) being transported through the tubing. In addition, the polyolefin inner layer resists changes to the tubing itself by the transport of medicinal fluid therethrough.

In some embodiments, the continuous inner layer can comprise or consist of a functionalized polyolefin such as a maleic anhydride modified polyethylene (e.g., a high density polyethylene HDPE or a low density polyethylene LDPE), a maleic anhydride modified polypropylene, maleic anhydride modified plastomer, or a combination thereof. Such functionalized polyolefins facilitate interlayer adhesion bonding between the inner and outer layers.

Advantageously, the inner layer is more rigid, e.g., stiffer, than the outer layer. Such a configuration facilitates solvent bonding of the medical tubing to other medical components. For example, the inner layer can have a tensile modulus in the range of about 1800 to about 3000 MPa such as between about 1000 MPa and 2000 MPa. Tensile stress at yield (MPa) can be determined by DIN 53 504, elongation at break (%) by DIN 53 504 and Shore D by DIN 53 505.

The outer layer can comprise or consist of a polymeric material different from the inner layer such that the outer surfaces of the tubing can have different properties from the inner layer materials. For example, the outer can comprise a thermoplastic polymer or a blend thereof such as, one or more or blend including, a thermoplastic polyurethane (TPU), a thermoplastic olefin (TPO), a thermoplastic elastomer (TPE), a styrene-containing thermoplastic elastomer (S-TPE), a polyolefin elastomer (POE), a styrenic blocking copolymer (SBC). The thermoplastic polymers useful as outer layers can further be blended with other polymeric components and/or additives. For example, thermoplastic polymers useful as outer layers can further be blended with adhesion promoters, such as acrylic based TPEs, or polar functionalized polyolefins, such as up to 15 wt % or more of the blend, tackifiers, and clarifying agents, such as up to 10 wt % of the blend. Many of the thermoplastic polymers have advantageous properties for the outer layer of medical tubing such as thermoplastic polyurethanes (TPU) and styrene-containing thermoplastic elastomers (S-TPE), which can be useful in solvent bonding the outer layer of the tubing. Alternatively, or in combination, many of the thermoplastic polymers advantageously improve flexibility of the tubing.

In some aspects of the medical tubing of the present disclosure, a tie layer can be included between the continuous inner layer and continuous outer layer.

Advantageously, the medical tubing of the present disclosure does not include polyvinyl chloride. That is, the outer layer and/or the inner layer and/or tie layer, if present, does not include polyvinyl chloride.

In some aspects, the medical tubing of the present disclosure can have a Shore A hardness of greater than about 85 or less than about 85. Hardness of Shore A greater than about 85 for medical tubing application is typically considered hard. Pump tubing typically employs softer tubing having a Shore A hardness of less than about 65, e.g., 55 or less.

Medical tubing of the present disclosure can be used as medical tubing for administration of medical fluid by infusion such as with intravenous assemblies, gravity containers and/or infusion pumps for the transport of intravenous fluid to a patient. An assembly of tubing, valves, fittings, and needles that connect a fluid container to a patient intravenously may be referred to as an “IV set”. Infusion pumps are medical devices that may be used to administer intravenous (IV) fluids. Such assemblies, containers and pumps employ tubing bound to one or more medical connectors and tubing of the present disclosure is useful as such.

For example, tubing according to the present disclosure can be readily mated and bonded to a medical connector or other medical component by applying a thin layer of solvent on one or both of the contacting surfaces and then the two surfaces are brought together and held in place until a seal is formed therebetween. The contacting surface of the tubing can be the outer surface of the outer layer and the contacting surface of the medical connector can be a pocket or inner surface of the connector. Medical connectors that can benefit from tubing of the present disclosure include, without limitation, connectors made of acrylic-based polymeric materials such as acrylonitrile-butadiene-styrene (ABS), methyl methacrylate-acrylonitrile-butadiene-styrene (mABS), acrylic-polycarbonate based materials, polyester, polycarbonate, acrylic, polyvinylchloride, etc.

In an embodiment of the present application, medical tubing according to the present disclosure can be readily bonded to a medical connector by solvent bonding. As described in the preceding paragraph, solvent boding includes applying a thin layer of solvent on one or both of the medical tubing and the medical connector. For example, a thin layer of solvent can be applied on outer surface of the medical tubing and the tubing inserted in an inner surface (e.g., a pocket or other element) of the medical connector or a thin layer of solvent can be applied in an inner surface of a medical connector and the tubing inserted in an inner surface, or both. The two surfaces (e.g., the tubing surface and the connector surface) one or both of which have the applied solvent are then brought together and held in place until a seal is formed therebetween.

Solvents that can be used in solvent bonding tubing of the present disclosure include, without limitation, one or more of an alcohol, such as methanol, ethanol, isopropanol, propanol, butanol, pentanol, benzyl alcohol, a glycol such as diethylene glycol, propylene glycol, an ether, such as dioxolane, tetrahydrofuran, 1,3-dioxane, a ketone, such as acetone, methyl ethyl ketone, cyclohexanone, an ester such as ethyl acetate, ethyl formate, an amide such as dimethyl formamide, dimethyl and diethyl acetamide, N-methyl pyrrolidone, a sulfoxide, such as dimethyl sulfoxide, a hydrocarbon such as hexanes, toluene, a chlorinated solvents such as methylene chloride, etc.

Table 1 below provide example of a medical tubing construction that can be used according to the present disclosure. Such medical tubing constructions and material formulations can meet many of the demands of an IV set and other demands of medical tubing.

TABLE 1
Solvent Bondable, Low Sorbing Polyolefin
Lined TPU Medical Tube Construction.

| Outer Layer - | Inner Layer - |
|---|---|
| Thermoplastic Polyurethane | Functionalized Polyolefin |
| Tecoflex EG-85A (1) or | Toyo-Tac M-100 (5) |
| Pellethane 2363-80A (2) or | Exxelor PE1040 (6) |
| Ellastollan 1180A (3) or | TecnoBond PE-LMP (7) |
| Tecothane TT1095A (4) TPU | Exxelor VA1840 (8) |
| Tecoflex EG-85A or | Custom made amine functionalized |
| Pellethane 2363-80A or | polyolefin through reactive extrusion: |
| Ellastollan 1180A or | choice of Functionalized Polyolefin |
| Tecothane TT1095A TPU | 1,2,3,4 above reacted with polyether |
| | amines |

(1) Tecoflex EG-85A is a TPU aliphatic polyether
(2) Pellethane is an aromatic polyether based polyurethane
(3) Ellastollan 1180A is an ether based thermoplastic urethane
(4) Tecothane TT1095A is a Shore A 95 TPU, which may satisfy different needs for small bore tubes.
(5) Toyo-Tac M-100 is a maleic anhydride PP
(6) Exxelor PE1040 is a maleic anhydride modified HDPE
(7) TecnoBond PE-LMP is a maleic anhydride PE
(8) Exxelor VA1840 is a maleic anhydride modified plastomer

As noted in Table 1, a continuous inner layer can comprise or consist of an amine functionalized polyolefin or maleic anhydride modified polyolefin. Such amine functionalized polyolefin can be prepared through reactive extrusion by combining and reacting a maleic anhydride modified polyolefin (e.g., choice of 1, 2, 3, 4 from Table 1 above) with a polyether amine. This formulation or maleic anhydride functionalized polyolefin allows increased compatibility of the polyolefin, a non-polar low surface energy material, with a more polar material group. Tube architecture in Table 1 requires good interlayer adhesion between a polar and non-polar material group, hence this customization should increase adhesion of the inner layer to the outer layer.

The two-layer polyolefin lined TPU medical tubing construction as provide in Table 1 offers several advantages, including: lack of a trilayer approach, increase in the processing line speed/line output compared with a trilayer, e.g., coextrusions run should be faster and less complicated than a trilayer. Standard cost is expected to be lower than trilayer, good adhesion of layers due to compatibilization via functionalization of the inner layer compared with poor adhesion of a non-modified polyolefin as a result of the functionalized polyolefin inner layer. Polyolefins further advantageously have low drug sorbing properties.

Further in some embodiments, the continuous inner layer can be relatively thinner than the continuous outer layer which should encourage the tubing to have similar bulk properties of the continuous outer layer such as a TPU outer layer. In addition, ether based TPU layers provide better solvent bondability, and the choice of an aliphatic backbone provides better color stability under certain circumstance.

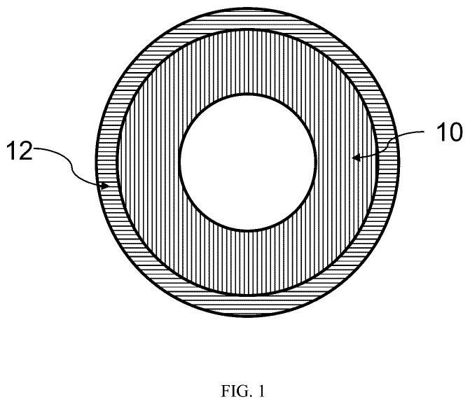

FIG. 1 illustrates an example of a two-layer polyolefin lined medical tube. Such a construction can be used with the polymeric materials listed in Table 1 above to provide a solvent bondable, low sorbing double layer polyolefin lined TPU medical tube. As shown, the two-layer medical tube includes as inner layer (10) and an outer layer (12) directly on the inner layer.

For use in applications including IV sets and/or infusion pumps, tubing of the present disclosure can have an inner diameter for flow of fluid therethrough ranging from about 1.5 mm to about 6 mm, e.g., from 2 mm to 4 mm. The overall sidewall thickness can range from about 0.2 mm to about 1 mm, such as from 0.4 mm to 0.6 mm. In some aspects of the present disclosure, the outer layer can comprise 10 to 90% of the side wall thickness, the inner layer can comprise 90 to 10% of the sidewall thickness. In an embodiment, the outer layer can have a thickness of about 0.01 mm to about 0.5 mm, e.g., from about 0.05 mm to about 0.2 mm, and the inner layer can have a thickness of about 0.01 mm to about 0.8 mm, e.g., from about 0.02 mm to about 0.1 mm such as about 0.038 mm (about 0.0015 inches).

Table 2 below is another example of a medical tubing construction that can be used according to the present disclosure.

TABLE 2
Solvent Bondable Low Sorbing PE
Lined TPU Tube with a Tie Layer

| Outer Layer - | | |
|---|---|---|
| Thermoplastic | | Inner Layer |
| Polyurethane | Tie Layer | Polyolefin |
| Tecoflex EG-85A or | TecnoBond PP-TLA (1) | 23R2A (2) |
| Pellethane 2362-80A | EC800AA (4) | EF612 MFI 2.5 (3) |
| or Ellastollan | REPSOL | EF612 MFI 2.5 (3) |
| 1180A | HEALTHCARE ® | |
| | HVA18G (5), | |
| | see (6) | |

(1) TecnoBond PP/TLA is a maleic anhydride modified random block PP copolymer
(2) Random PP copolymer extrusion grade
(3) LDPE
(4) Low-density polyethylene EC800 is a general-purpose low-density formulation used for extrusion coating applications.
(5) EVA copolymer
(6) Other alternatives include ReZilok Rx as a linear low-density polyethylene grafted with maleic anhydride.

As provided in Table 2, a medical tube can include a continuous inner layer, a continuous outer layer thereon and a tie layer between the continuous inner layer and continuous outer layer. As provided in Table 2, the inner layer can comprise a polyolefin such as a polypropylene or polyethylene (e.g., linear low-density polyethylene, LDPE). The outer layer can comprise a thermoplastic polyurethane (TPU), which can be useful in solvent bonding the outer layer of the tubing. The medical tubing can also include a tie layer between the continuous inner layer and continuous outer layer. Such a tie layer can comprise a functionalized polyolefin such as a maleic anhydride modified polypropylene, a maleic anhydride modified polyethylene, or other tie layer polymers such as an ethyl vinylacetate copolymer (EVA copolymer), or combinations thereof.

The three-layer polyolefin lined TPU medical tubing construction as provide in Table 2 offers several advantages, including: compatible backbone selection matching PP with PP based TPO or POP, matching PE with PE based TPO or POE enable compatibility without the need of a more expensive functionalized polymer, low drug sorbing property with inclusion of PP or PE based inner layer.

Further in some embodiments, the continuous inner layer can be relatively thinner than the continuous outer layer which should encourage the tubing to have similar bulk properties of the TPU layer. In addition, ether based TPU layers provide better solvent bondability, and the choice of aliphatic backbone provides better color stability.

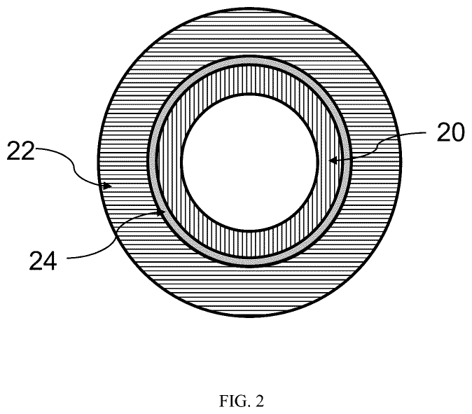

FIG. 2 illustrates an example of a three-layer polyolefin lined medical tube. Such a construction can be used with the polymeric materials listed in Table 2 above to provide a solvent bondable, low sorbing polyolefin lined TPU medical tube with a tie layer. As shown, the three-layer medical tube includes an inner layer (20), an outer layer (22) and a tie layer (24) between and directly contacting inner layer (20) and outer layer (22).

For use in applications including IV sets and/or infusion pumps, tubing of the present discloser can have an inner diameter for flow of fluid therethrough ranging from about 1.5 mm to about 6 mm, e.g., from 2 mm to 4 mm. The overall sidewall thickness can range from about 0.2 mm to about 1 mm, such as from 0.4 mm to 0.6 mm. In some aspects of the present disclosure, the outer layer can comprise 10 to 90% of the side wall thickness, the inner layer can comprise 90 to 10% of the sidewall thickness. In an embodiment, the outer layer can have a thickness of about 0.1 mm to about 0.8 mm, e.g., from about 0.5 mm to about 0.5 mm, and the inner layer can have a thickness of about 0.01 mm to about 0.5 mm, e.g., from about 0.05 mm to about 0.2 mm.

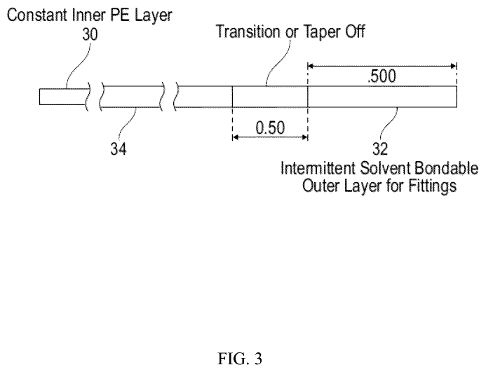

FIG. 3 illustrates a multi-material, polyolefin lined medical tube. Such a construction can be used to provide a solvent bondable, low sorbing polyolefin lined medical tube. As shown, the medical tube includes a continuous inner polyolefin layer (30) and a continuous outer layer (34). For this example, the medical tubing further includes an intermittent solvent bondable segment layer (32) directly contacting outer layer 34. The intermittent solvent bondable segment layer can comprise a thermoplastic polyurethane such as a TPU with good bondability.

Table 3 below is another example of a medical tubing construction that can be used according to the present disclosure.

TABLE 3
Low Sorbing PE lined TPO Two-Layer Tube.

| Outer Layer | Inner Layer |
|---|---|
| Engage 8003 (1) | LDPE, HDPE, other ethylene based polyolefins |
| Engage 8452 (2) | LDPE, HDPE, other ethylene based polyolefins |
| Versify 3300 (3) | Random PP copolymer, sterilization grade |
| Vistamaxx 3020 (4) | Random PP copolymer sterilization grade |

(1) Ethylene-octene polyolefin elastomer (POE)
(2) Ethylene-butene polyolefin elastomer (POE)
(3) POP
(4) Propylene elastomer, i-PP repeat units with random ethylene

As provided in Table 3 above, a medical tube can include a continuous inner layer and a continuous outer layer directly thereon. The continuous inner layer can comprise a non-functionalized polyolefin such as polyethylene (e.g., a LDPE, HDPE) or polypropylene. The continuous outer layer can comprise a thermoplastic polymer such as an polyolefin elastomer (POE), e.g., an ethylene-octene polyolefin elastomer, ethylene-butene polyolefin elastomer, a polyolefin plastomer (POP), a propylene elastomer, as well as other thermoplastic polymers or blends thereof.

Many thermoplastic polymers including thermoplastic elastomers (TPE) and thermoplastic olefins useful for the outer layer of medical tubing of the present disclosure can be compounded with polyolefins for cost optimization, increased crystallinity, increased mechanical strength, increased working range, i.e., environmental stability and shelf life, and increased material compatibility, i.e., interlayer adhesion to the inner layer. Below are a few different customizations that can overcome certain deficiencies of thermoplastic polymers from s-TPE and TPOs.

Custom made styrenic blocking copolymer (SBC) compounds: SBC grades are useful due to their flexibility as well as solvent response, i.e., bondability. However, some of our experiments indicate inferior kink resistance than other polymer types within the styrenics TPEs. Moreover, certain SBC grades lack the high temperature resistance as observed through low softening points. Similarly, POP and POE grades such as Engage reactor made TPOs lack the high temperature resistance as observed through low softening points. Tables 4-5 provides examples of thermoplastic polymer blends that can be used as a continuous outer layer of medical tubing according to the present disclosure.

SBC blend with random PP copolymer: An SBC blend with random PP copolymer will create a solvent bondable kink resistant material for tubing as-is or for solvent bondable layering. The blend ratios may vary from 5 to 95% of olefinic component (ethylene or propylene based) addition as to be further refined based on transparency requirement. These blends may also contain adhesion promoters such as acrylic based TPEs, or polar functionalized polyolefins up to 15 wt % (of the blend). Addition of tackifiers and clarifying agents, and polymer processing aids up to 10 wt % (of the blend) is typical for these types of compound when needed to modify the melt flow rheology and transparency of the tubing. Various processing variables can impact the transparency of the tubing as well as its mechanical properties.

TPO blend with random PP copolymer, polyolefins or temperature resistant TPOs such as Olefinic Block copolymers. The blend ratios may vary from 5 to 95% of olefinic component (ethylene or propylene based) which can be adjusted based on transparency requirement. These blends may also contain adhesion promoters such as acrylic based TPEs, or polar functionalized polyolefins up to 15 wt % of the blend. Addition of tackifiers and clarifying agents and polymer processing aids up to 10 wt % (of the blend) is typical for these types of compound when needed to modify the melt flow rheology and transparency of the tubing. Various processing variables can impact the transparency of the tubing as well as its mechanical properties.

TABLE 4
Examples of custom TPEs blends

| Function | Compound A | Compound B | Compound C |
|---|---|---|---|
| Elastomeric for | Engage 8003 | Engage 7256 | Engage 8452 |
| flexibility, clarity | | | |
| High Temperature | Ethylene based | Ethylene based | Ethylene based |
| Stability, Drug | Polyolefin, LDPE, | Polyolefin, LDPE, | Polyolefin, LDPE, |
| Compatibility (Low | HDPE or alike | HDPE or alike | HDPE or alike |
| Sorbing of most | | | |
| grades), Cost | | | |
| Optimizer (Most | | | |
| Grades) | | | |
| Adhesion, Rheology | Acrylic based | Acrylic based | Acrylic based |
| Modifiers | TPEs, Polar | TPEs, Polar | TPEs, Polar |
| | functionalized | functionalized | functionalized |
| | polyolefins, | polyolefins, | polyolefins, |
| | Tackifiers | Tackifiers | Tackifiers |
| Clarity Adjustment | Clarifying or | Clarifying or | Clarifying or |
| | Nucleating Agents, | Nucleating Agents, | Nucleating |
| | polymer processing | polymer processing | Agentspolymer |
| | aids, if needed | aids, if needed | processing aids, , |
| | | | if needed, |

TABLE 5
Additional examples of custom TPEs blends

| Function | Compound D | Compound E | Compound F |
|---|---|---|---|
| Elastomeric for | Engage 8003, POE | Infuse 9010 | Ineos 4G80 SBC |
| flexibility, clarity | | olefinic block | |
| | | copolymer (OBC) | |
| High Temperature | Infuse 9010, OBC | Random PP | Random PP |
| Stability, Drug | | copolymer | copolymer |
| Compatibility (Low | | | |
| Sorbing of most | | | |
| grades), Cost | | | |
| Optimizer (Most | | | |
| Grades) | | | |
| Adhesion, Rheology | Acrylic based | Acrylic based | Acrylic based |
| Modifiers | TPEs, Polar | TPEs, Polar | TPEs, Polar |
| | functionalized | functionalized | functionalized |
| | polyolefins, | polyolefins, | polyolefins, |
| | Tackifiers | Tackifiers | Tackifiers |
| Clarity Adjustment | Clarifying or | Clarifying or | Clarifying or |
| | Nucleating Agents, | Nucleating Agents, | Nucleating Agents, |
| | polymer processing | polymer processing | polymer processing |
| | aids, if needed | aids, if needed | aids, if needed |

Infuse 9010 olefinic block copolymer (OBC)
Engage 8003, Engage 7256, and Engage 8452 Polyolefin elastomer
Ineos 4G80 Styrene butadiene block copolymer

A two-layer polyolefin lined thermoplastic polymer medical tubing construction, such as provide in Table 3, offers several advantages, including: lack of a trilayer approach, increase in the processing cycle time compared with a trilayer, e.g., coextrusions run should be faster and less complicated than a trilayer, cost is expected to be lower than trilayer, good adhesion of layers due to compatibilization with the inner layer, low drug sorbing property with inclusion of non-functionalized polyolefin inner layer.

Advantageously, the medical tubing according to the present disclosure has a bond strength that is at least 25% higher, such as at least 50%, 75% and 100% higher, relative to medical tubing comprised of a monolayer of the thermoplastic polymer. To show the bond strength of medical tubing of the present disclosure, several medical tubing configurations were prepared and compared to medical tubing composed solely of the thermoplastic polymer but otherwise having the same dimensions of a two layer medical tubing (referred to as a “mono” tube). Since the polyolefin inner layer does not undergo significant, if any, solvent bonding, there was no need to compare the medical tubing of a monolayer of the polyolefin material.

The medical tubing provided in Tables 6A and 6B below were solvent bonded to a luer connector made of mABS Terlux® 2802HD. The maximum force needed to break the tube/connection assembly was recorded on an Instron with a pull rate of 20 inches/minute (bond strength). Table 6C provides some physical properties of certain inner layers.

TABLE 6A
Tubing Architectures Including Double Layers or Monolayer Tubes

| Designation | Trial No. | Outer Layer | Inner Layer |
|---|---|---|---|
| Mono-PB | 1 | RB820 | None |
| | | (Polybutadiene) | |
| Mono-TPO1 | 3 | Engage 8003 | None |
| | | (TPO post-reactor) | |
| Mono-TPO6 | 6 | Vistamaxx 3020 | None |
| | | (TPO post-reactor) | |
| Mono-TPU1 | 2 | Estane 58271 | None |
| | | (TPU aromatic | |
| | | polyester) | |
| Mono-TPU2 | 9 | Tecoflex EG-85A | None |
| | | TPU aliphatic | |
| | | polyether | |
| PE1 Lined | 15 | Tecoflex EG-85A | Toyo-Tac M100 |
| TPU2 | | (TPU aliphatic | (Modified |
| | | polyether) | polyolefin) |
| PE2 Lined | 16 | Tecoflex EG-85A | Exxelor PE1040 |
| TPU2 | | (TPU aliphatic | (Modified HDPE) |
| | | polyether) | |
| PE3 Lined | 22 | PUR 9741 | PE140/PE770 |
| TPU3 | | (TPU polyether) | (LDPE/EVA) |
| Mono-TPE1 | 10 | 4G80 | None |
| | | (SBC) | |
| Mono-TPE2 | 8 | PP 6649 | None |
| | | (styrenic based TPE) | |
| Mono-TPE3 | 23 | Medalist MD50278 | None |
| | | (styrenic based TPE) | |
| Double-TPE3 | 14 | Hexpol 880210 | Medalist MD50278 |
| | | (SBC) | (STPE) |
| PE-Lined- | 20 | PP 6469 | PE140/PE770 |
| TPE2 | | (styrenic based TPE) | (LDPE/EVA) |

TABLE 6B
Tubing Architecture of an

| Designation | Trial No. | End Segment | Mid-Segment |
|---|---|---|---|
| AET-TPE3 | 5 | Hexpol 880210 | Medalist MD50278 (sTPE) |
| | | (SBC) | |

TABLE 6C

| | Exxelor PE1040 | Toyo-tac | Raumedic PE140 |
|---|---|---|---|
| Density | 0.96 | 0.9 | 0.9 |
| Melt Index, | 1.4 [190/2.16] | 7 [190/2.16] | NA |
| g/10 min | | | |
| Tm, C. | 132 | 150 | NA |
| Shore D, | NA | NA | 45 +/− 3 |
| DIN53 505 | | | |

Notation for melt index: [190/2.16] = 190 C, and 2.16 kg

The data in Table 6B was determined using DIN 53 479 (Density, g/cm3) and hardness can be determined using Shore D (DIN 53 505), tensile stress at yield (MPa) (DIN 53 504), elongation at break (%)(DIN 53 504). The Inner Layer Wall thickness/Total Wall Thickness was about 20%, and Outer Later Wall thickness/Total Wall Thickness was about 80%.

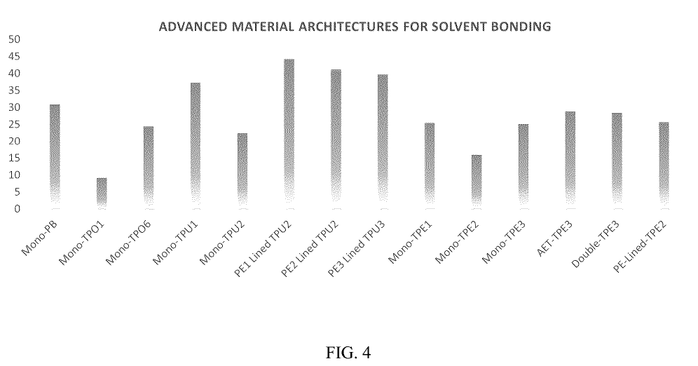

The results of the Instron testing is shown in FIG. 4. As shown in FIG. 4, the maximum load (Newtons) needed to break the assembly (bond strength) of medical tubing bound to the medical connector (e.g., a luer) was significantly higher for medical tubing having a polyolefin lined thermoplastic polymer compared to medical tubing comprised of a monolayer (“mono”) of a polymeric material (control). This is shown by comparing PE1 Lined TPU2 and PE2 Lined TPU2 (polyolefin lined thermoplastic polyurethanes), which had bond strengths of about 44 and 42 N, respectively, to Mono-TPU2, which had a bond strength of about 22 N.

The medical tubing having a polyolefin lined thermoplastic polymer had about a 100%-91% higher bond strength relative to a medical tube comprised of a monolayer of the thermoplastic polymer. The same trend is demonstrated with a comparison of a polyolefin lined styrenic based thermoplastic elastomer (PE-Lined TPE2) to a monolayer styrenic based thermoplastic elastomer (Mon-TPE2). As shown in FIG. 4, the polyolefin lined TPE has a higher bond strength by about 67%. Medical tubing prepared by alternative extrusion technology (AET) also showed an increase in bond strength

The outer layer of the tubing allows solvent bonding of the tubing to pocket joints of medical connectors such as connectors comprising rigid acrylic-based materials. It is believed that when the inner layer is more rigid, e.g., stiffer, than the outer layer, such a configuration facilitates solvent bonding of the medical tubing to other medical components.

The medical tubing of the present disclosure can be manufactured by extrusions. For example, medical tubing according to the present disclosure can be coextruded as a continuous inner layer having a continuous outer layer thereon, in which the inner layer comprises a polyolefin and the outer layer comprises a polymeric material that is different from the inner layer. It is preferable to make medical tubing that is clear, e.g., transparent to visible light. Extruding clear tubing has a set of challenges; the processing variables below can make a difference between transparent vs hazy tubing. Clarity is impacted by die temperatures, tooling design, and parison ventilation.

Die Temperatures: (a) Die temperatures will have an effect on the transparency of the tubing through its interaction with the surface finish of the tubing. Lowering the die temperatures near the end of the die will cause the outermost layer (the surface) of the tubing to “stick” to the tooling as its extruded. The sticking will cause a differential in velocity of the polymer melt from the inside to the outside, and will cause the surface to have micro deformations (“rips”) on the surface. These rips cause the tubing to have an opaque or “frosted” appearance. (b) Conversely, increasing the die temperatures will serve to make the tubing more transparent by reducing the differential in polymer melt velocity from the inside to the outside, causing less micro deformations to appear on the surface.

Tooling Design: (a) Extrusion tooling can have a similar effect on surface finish as die temperatures does. By lowering the friction between the tooling material and the polymer melt, the polymer melt velocity would be more consistent throughout the parison. This reduction in friction is achieved through applying a finishing or coating process to the tooling.

Parison Ventilation: (a) One last process parameter that may have an effect on tubing transparency would be the placement of the parison vent. The purpose of the vent is to ventilate any fumes from the parison as it is extruded, and it is usually placed directly above the parison. A vent with a large opening can be placed farther away from the parison to catch any fumes, but a small snake type vent would have to be placed within inches of the parison. If the vent is placed too close to the parison, the parison would be affected by the suction of the air, and this would alter the surface finish of the tubing, potentially creating an opaque/frosted finish. (b) This is similar to a frosting extrusion process, where chilled air is blown onto the parison directly as it is extruded.

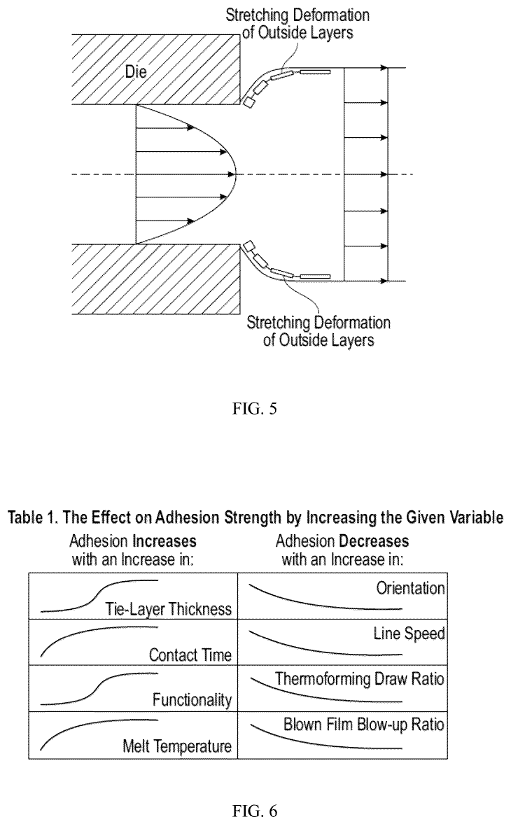

FIG. 5 illustrates how items can be affected during extrusion and FIG. 6 shows the effects of adhesion strength by adjusting certain variables during extrusion.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. Medical tubing comprising a continuous inner layer having a continuous outer layer thereon;
wherein the inner layer comprises a polyolefin;
wherein the outer layer comprises a thermoplastic polymer selected among a thermoplastic polyurethane (TPU), a thermoplastic olefin (TPO), a thermoplastic elastomer (TPE), a styrene-containing thermoplastic elastomer (S-TPE), a polyolefin elastomer (POE), a styrenic blocking copolymer (SBC); and
wherein the medical tubing has a bond strength that is at least 25% higher relative to a medical tube comprised of a monolayer of the thermoplastic polymer.

2. The medical tubing of claim 1, wherein the inner layer consists of the polyolefin and the outer layer consists of a thermoplastic polymer.

3. The medical tubing of claim 2, wherein the polyolefin consists of a polyethylene or a polypropylene.

4. The medical tubing of claim 2, wherein the polyolefin consists of a maleic anhydride modified polyethylene, a maleic anhydride modified polypropylene, or a combination thereof.

5. The medical tubing of claim 2, wherein the thermoplastic polymer consists of a thermoplastic urethane.

6. The medical tubing of claim 1, wherein the thermoplastic polymer is one or more of, or a blend including a styrene-containing thermoplastic elastomer (S-TPE), a polyolefin elastomer (POE), a styrenic blocking copolymer (SBC).

7. The medical tubing of claim 1, wherein the outer layer and inner layer do not include polyvinyl chloride.

8. The medical tubing according to claim 1, wherein the inner layer directly contacts the outer layer.

9. The medical tubing according to claim 1, wherein the medical tubing has a Shore A hardness of less than about 65.

10. An infusion set comprising the medical tubing of claim 1 bound to a medical connector via solvent bonding.

11. The infusion set of claim 10, wherein the medical connector comprises an acrylic-based polymer and the medical tubing is bound to the acrylic-based polymer.

12. The infusion set of claim 10, wherein the inner layer consists of the polyolefin and the outer layer consists of a thermoplastic polymer.

13. The infusion set of claim 12, wherein the polyolefin consists of a polyethylene or a polypropylene and the thermoplastic polymer consists of a thermoplastic urethane.

15. A method of preparing an infusion set comprising the medical tubing of claim 1, the method comprising:
applying a thin layer of a solvent on one or both of a surface of the medical tubing and a surface of a medical connector, and
after applying the thin layer of the solvent, holding the surface of the medical tubing and the surface of the medical connector together until a seal is formed therebetween.

16. The method of claim 15, wherein the solvent is applied to the outer layer of the tubing as the surface of the tubing.

17. The method of claim 15, wherein the medical connector comprises an acrylic-based polymer.

18. The method of claim 15, wherein the solvent includes one or more of an alcohol, a glycol, a ketone, an ester such as ethyl acetate, an amide a sulfoxide, or a hydrocarbon.

19. The method of claim 15, wherein the inner layer consists of the polyolefin and the outer layer consists of a thermoplastic polymer.

20. The method of claim 19, wherein the polyolefin consists of a polyethylene or a polypropylene.
